(19) United States
(12) Patent Application Publication
(10) Pub. No.: US 2026/0311851 A1
(43) Pub. Date: Oct. 8, 2026

(54) System and Method for Continuously Reallocating Heterogeneous Evacuation Assets Across Dispatching Subsystems

(71) Applicant: Mahdi Al-Husseini, Coffee, AL (US)

(72) Inventor: Mahdi Al-Husseini, Coffee, AL (US)

(73) Assignee: UNITED STATES GOVERNMENT AS REPRESENTED BY THE SECRETARY OF THE ARMY, Adelphi, MD (US)

(21) Appl. No.: 19/098,323

(22) Filed: Apr. 2, 2025

(51) Int. Cl. G16H 40/20

(52) U.S. Cl. CPC G16H 40/20

(57) ABSTRACT

An evacuation manager that implements continuous reallocation of heterogeneous evacuation assets across dispatching subsystems is described. The intelligent organization and utilization of heterogeneous evacuation assets across potentially non-contiguous battlefields during evolving high-intensity combat considers critical factors including, for example, flight regulation requirements, historical casualty flows, evacuation asset characteristics, and/or other relevant factors. Associated dynamic resource allocation models quickly become intractable when integrating even a reduced number of evacuation assets in select fixed locations, given the various combinations of aircraft, aircrews, and evacuation request execution strategies. The evacuation manager implements a three-stage hierarchical framework with structural, high-level, and low-level planners, each using, for example, a Markovian model with Monte Carlo search solutions

### GOVERNMENT INTEREST

The invention described herein may be manufactured, used and licensed by or for the U.S. Government.

### BACKGROUND OF THE INVENTION

Future conflicts with near-peer actors are expected to produce orders of magnitude more casualties than traditional conflicts. Existing medical planner dispatch policies are inadequate and unable to handle hundreds of casualties or more per day in constrained environments.

Thus there is a need for a data-centric approach is formulated through the proposed invention, which is flexible in nature and may be adapted to any environment or evacuation asset type.

### BRIEF SUMMARY OF THE INVENTION

Some embodiments provide an evacuation manager that implements continuous reallocation of heterogeneous evacuation assets (e.g., aircraft, watercraft, ground vehicles, etc.) across dispatching subsystems. The evacuation manager may include or utilize a high-level planner that allocates evacuation requests to dispatching subsystems. The evacuation manager may include or utilize a low-level planner associated with each dispatching subsystem that dispatches potentially heterogeneous evacuation assets to allocated evacuation requests. The evacuation manager may include or utilize a structural planner that continuously reallocates evacuation assets across dispatching subsystems. Reinforcement learning techniques and information sharing among the system components may be used to develop policies that inform high-level, low-level, and/or structural planners.

The evacuation manager of some embodiments enables dynamic and efficient evacuation operations in a variety of settings by introducing a hierarchical system of planners and policies designed to support a heterogeneous mix of evacuation assets. There are three levels of planners in some embodiments-high-level, low-level, and structural. Each structural planner continuously reallocates evacuation assets across dispatching subsystems. Each high-level planner allocates evacuation requests to the individual dispatching subsystems, which may be associated with a low-level planner to dispatch aircraft and crews to those evacuation requests in a manner respecting aviation and crew requirements.

The intelligent organization and utilization of heterogeneous evacuation assets across potentially non-contiguous battlefields during evolving high-intensity combat considers critical factors to include flight regulation requirements, historical casualty flows, and evacuation asset characteristics. Associated dynamic resource allocation models quickly become intractable when integrating even a reduced number of evacuation assets in select fixed locations, given the various combinations of aircraft, aircrews, and evacuation request execution strategies. The three-level hierarchical framework may include structural, high-level, and low-level planners.

Each planner may use a Markovian decision process (MDP) with stochastic search solutions (and/or other model formulations and/or algorithms may be applied).

Future military conflicts will involve near-peer threats possessing significant warfighting capabilities resulting in high casualty counts. The increased number of casualties will drastically change how evacuation procedures are facilitated from the theater-level down to the lowest echelons. Evacuation assets will be significantly taxed. It is therefore especially important that data is used to inform how those assets are utilized such that patient outcomes in aggregate are optimized. The hierarchical model introduced uses historical and simulated data to best allocate, reallocate, and dispatch assets to requests at scale.

The optimization of evacuation asset utilization has been demonstrated to have tremendous value in the civilian emergency medical services (EMS) sector. The evacuation manager of some embodiments provides a framework enabling various software systems (including legacy systems), at scale, and in support of multiple kinds of assets (e.g., ground vehicles, aircraft, watercraft, etc.).

The coordination of multiple heterogeneous medical evacuation assets across long distances and multiple types of terrain can quickly become a computationally intractable problem, further complicated by uncertainty associated with evolving evacuation flows. Such coordination may include the use of tail-to-tail transfers at the boundaries between different types of terrain. The policies governing each of these evacuation assets (e.g., aircraft, watercraft, ground vehicles, etc.), the endurance of their crews, their locations in three-dimensional space, and cruising speeds, all may affect the determination whether to dispatch an evacuation asset to an allocated request. Additionally, the bed utilization at different treatment facilities must be considered to prevent overcrowding. Certain patients may need to be delivered to certain kinds of treatment facilities based on, for example, injury type and/or facility capabilities.

Dynamic resource allocation (DRA) involves the fluid reorganization of resources such as evacuation assets in pursuit of evolving requirements such as dynamic casualty flows. DRA problems may typically be modeled as MDPs which support the identification of optimal policies relating agent actions to system states. MDPs may be multi-agent in nature (e.g., associated with multiple aircraft, watercraft, and/or ground vehicles), with either perfect or imperfect information sharing between agents. When agent actions are asynchronous due to temporal transitions, a semi-Markovian model may be used. Agent actions may then be considered once the last assigned action is fully resolved (referred to as asynchronous termination). These characteristics suggest that medical evacuation asset organization and utilization problems, which are inherently intertwined, are best modeled as multi-agent semi-Markovian decision processes (MSMDPs). The intractable nature of many MSMDPs modeling real-world problems may be overcome using hierarchical planning, in which a larger problem is divided into smaller sub-problems. Thus, two optimization problems are encoded in the hierarchy-how to optimally divide the larger problem into smaller sub-problems, and how to optimally allocate resources to and through the identified sub-problems.

### BRIEF DESCRIPTION OF DRAWINGS

The novel features of the disclosure are set forth in the appended claims. However, for purposes of explanation, several embodiments are illustrated in the following drawings.

FIG. 1 illustrates an example overview of one or more embodiments described herein, in which an aircraft is reallocated to a dispatching subsystem;

FIG. 2 illustrates an example overview of one or more embodiments described herein, in which exemplary evacuation operations are managed;

FIG. 3 illustrates an example hierarchical framework of one or more embodiments described herein;

FIG. 4 illustrates an example hierarchical framework of one or more embodiments described herein;

FIG. 5 illustrates a schematic block diagram of an environment of one or more embodiments described herein;

FIG. 6 illustrates a flow chart of an exemplary process that allocates evacuation requests;

FIG. 7 illustrates a flow chart of an exemplary process that allocates evacuation assets;

FIG. 8 illustrates another example overview of one or more embodiments described herein, in which exemplary evacuation operations are managed; and

FIG. 9 illustrates a schematic block diagram of one or more exemplary devices used to implement various embodiments.

### DETAILED DESCRIPTION OF THE INVENTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide an evacuation manager that implements continuous reallocation of heterogeneous evacuation assets across dispatching subsystems.

The intelligent organization and utilization of heterogeneous evacuation assets across potentially non-contiguous battlefields during evolving high-intensity combat considers critical factors including, for example, flight regulation requirements, historical casualty flows, evacuation asset characteristics, and/or other relevant factors. Associated dynamic resource allocation models quickly become intractable when integrating even a reduced number of evacuation assets in select fixed locations, given the various combinations of aircraft, aircrews, and evacuation request execution strategies. The evacuation manager implements a three-stage hierarchical framework with structural, high-level, and low-level planners, each using, for example, a Markovian model with Monte Carlo search solutions.

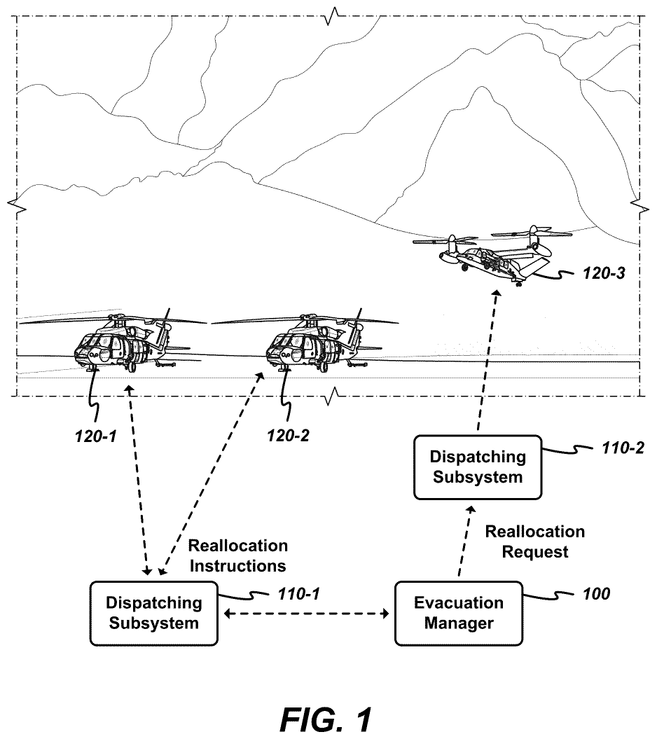

FIG. 1 illustrates an example overview of one or more embodiments described herein, in which an aircraft is reallocated to a dispatching subsystem. In this example, evacuation manager 100 may communicate with dispatching subsystems 110 to implement an evacuation plan. In this example, aircraft 120-1 and 120-2 may have been already allocated to dispatching subsystem 110-1 (e.g., based on an evacuation request received at evacuation manager 100).

Evacuation manager 100 may determine that additional resources are appropriate, and send a reallocation request indicating that aircraft 120-3 should be reallocated to dispatching subsystem 110-1, where aircraft 120-3 may be associated with a different dispatching subsystem 110-2. The reallocation request may be sent by evacuation manager 100 to the associated dispatching subsystem 110-2 associated with the reallocated aircraft 120-3, and the dispatching subsystem 110-2 may send a reallocation request (and/or other instructions) to aircraft 120-3. In some cases, the reallocation request may be sent from the evacuation manager 100 to the reallocated resource (i.e., aircraft 120-3 in this example).

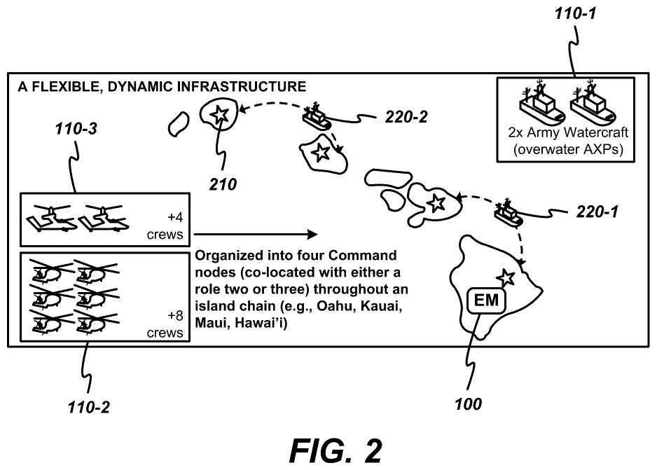

FIG. 2 illustrates an example overview of one or more embodiments described herein, in which exemplary evacuation operations are managed. In this example, simulated evacuation outcomes associated with a non-contiguous maritime environment are depicted. As shown, the evacuation manager 100 may interact with various dispatching subsystems 110 and/or associated watercraft 220 and/or aircraft 120.

Each dispatching subsystem 110 may be associated with any number of aircraft 120, watercraft 220, and/or ground vehicles (omitted for clarity). Each aircraft 120, watercraft 220, and/or ground vehicle may be able to interact with other components (e.g., via radio communication) and may receive commands or instructions from resources such as dispatching subsystem 110 and/or evacuation manager 100.

In this example, a set of command nodes 210 are distributed throughout an island chain (based on the Hawaiian islands). Evacuation manager 100 may coordinate and/or otherwise manage operations among the dispatching subsystems 110, command nodes 210, aircraft 120, and/or watercraft 220. Evacuation manager 100 is shown as being located on a particular island, but the evacuation manager 100 may be implemented at various appropriate locations and/or associated with various appropriate resources (e.g., one or more aircraft 120, watercraft 220, dispatching subsystems 110, and/or other appropriate elements such as servers, user devices, etc.). In some cases, the evacuation manager 100 may be implemented as a distributed system implemented across multiple devices, systems, components, etc. (e.g., the evacuation manager 100 may be distributed among the set of command nodes 210).

As shown, three dispatching subsystems 110 may be available. In this example, dispatching subsystem 110-1 may be associated with watercraft 220-1 and 220-2, dispatching subsystem 110-2 may be associated with some number of a first type of aircraft (e.g., aircraft 120-1) and a number of available crews, dispatching subsystem 110-3 may be associated with some number of a second type of aircraft (e.g., aircraft 120-2) and a number of available crews.

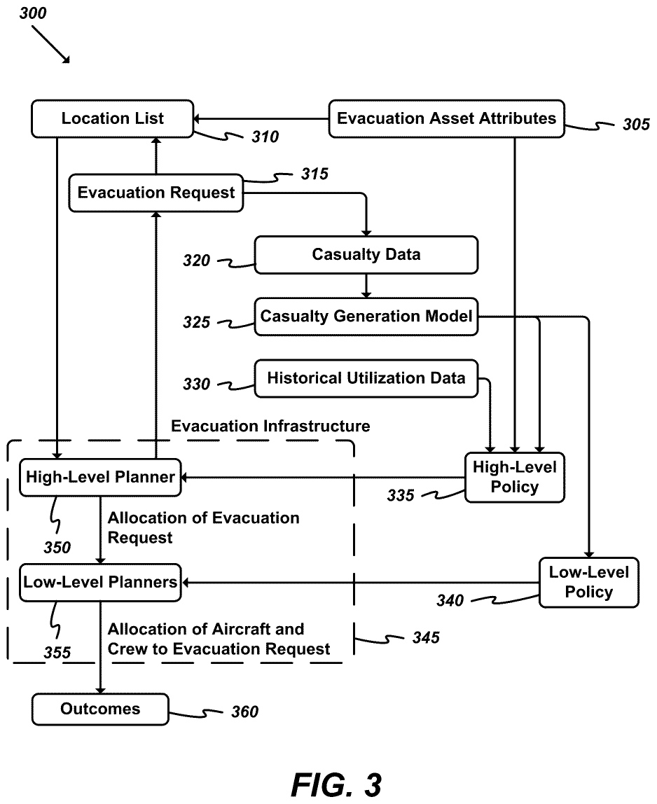

FIG. 3 illustrates an example hierarchical framework 300 of one or more embodiments described herein. Hierarchical framework 300 may be associated with task planning. Hierarchical framework 300 may be at least partially implemented by evacuation manager 100.

As shown, hierarchical framework 300 may include evacuation attributes 305, a location list 310, evacuation requests 315, casualty data 320, a casualty generation model 325, historical utilization data 330, one or more high-level policies 335, one or more low-level policies 340, an evacuation infrastructure 345 including one or more high-level planners 350 and one or more low-level planners 355, and a set of outcomes 360. Hierarchical framework 300 may be used to coordinate, for example, air and sea assets (e.g., aircraft 120, watercraft 220, dispatching subsystems 110, ground vehicles, etc.) to introduce a brand-new patient transfer capability using watercraft 220 as ambulance exchange points to double aeromedical evacuation capacity in noncontiguous maritime environments.

Evacuation asset attributes 305 may include information such as, for example, a listing of evacuation assets and associated asset information (e.g., type, location, speed, available patient carrying capacity, etc.). Evacuation asset attributes 305 may include an identifier or reference to an associated dispatching subsystem 110. Evacuation assets may include dispatching subsystems 110, aircraft 120, watercraft 220, ground vehicles, medical or other facilities, and/or other appropriate resources.

Location list 310 may be, or include, a discretized listing of evacuation location information.

Evacuation requests 315 may include request information as received from various external entities.

Casualty data 320 may include observed casualty data received from various evacuation assets and/or estimated or predicted casualty data. Casualty generation model 325 may include predicted casualty data based on modelling performed by the evacuation manager 100.

Historical utilization data 330 may include utilization data associated with transfer assets (e.g., dispatching subsystems 110, aircraft 120, watercraft 220, ground vehicles, medical or other facilities, and/or other appropriate resources).

Each high-level policy 335 may provide policy information to high-level planner 350 to implement allocation of evacuation requests to participating dispatching subsystems 110 and to determine tail-to-tail transfer locations.

Each low-level policy 340 may provide policy information to low-level planner 355 to implement, at or via the dispatching subsystem 110, allocation of assets and crew to the received evacuation requests 315. The low-level policy 340 may be used to inform the high-level policy 335 for optimal high-level planning.

Evacuation infrastructure 345 may be associated with a communication interface or similar element that allows interaction with the evacuation manager 100.

Each high-level planner 350 may implement allocation of evacuation requests based on one or more high-level policies 335.

Each low-level planner 355 may implement allocation of vessels and crew based on one or more low-level policies 340. In some cases, low-level planner 355 may be implemented autonomously by a resource such as dispatching subsystem 110.

The set of outcomes 360 may include aggregated patient outcome data (e.g., evacuation information, current status, prognosis, etc.).

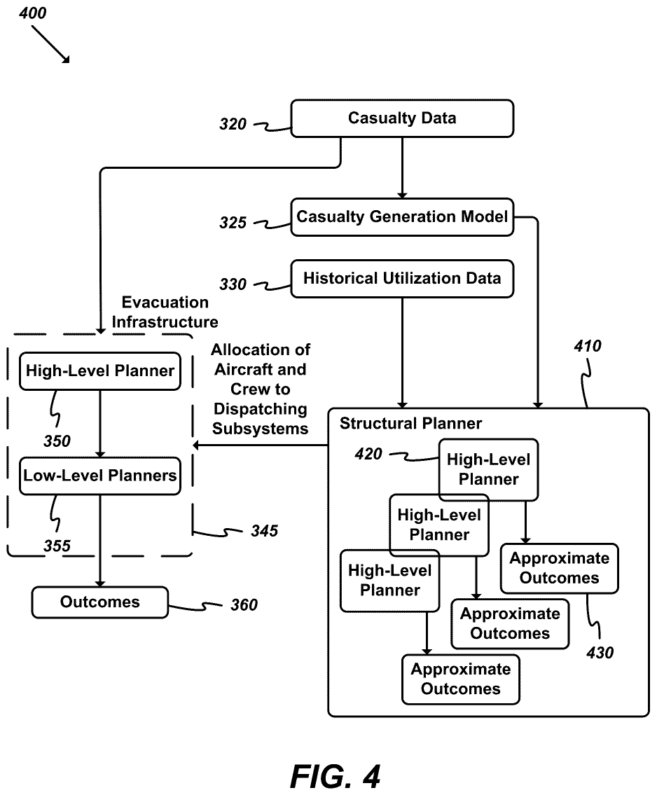

FIG. 4 illustrates another example hierarchical framework 400 of one or more embodiments described herein. Hierarchical framework 400 may be associated with task planning. Hierarchical framework 400 may be at least partially implemented by evacuation manager 100.

As shown, in addition to, and/or in place of, components described above in reference to hierarchical framework 300, hierarchical framework 400 may include a structural planner 410 including one or more high-level planners 420 and associated approximate outcomes 430.

Structural planner 410 may include any number of high-level planners 420 that may identify or generate high-level policies 335. The approximate outcomes 430 may be based on simulated and/or otherwise modeled data. Structural planner 410 may utilize casualty data 320, casualty generation model 325, and/or historical utilization data 330 to generate high-level policies 335 and/or determine approximate outcomes 430. The high-level planners 420 are configured to be surrogate models and therefore operate independently of low-level planners when estimating rewards, thus attaining a significant computational performance benefit. This permits the invention to consider large centralized multi-agent problems in real-time.

The hierarchical framework 400 may include a meta-planner that induces penalties for a hypothetical reorganization of assets and then simulates a variety of casualty chains over time to determine the expected impact to outcomes 360.

The hierarchical framework 400 may allow the evacuation manager 100 to redistribute evacuation assets dynamically over the course of a campaign in response to evolving casualty flows to optimize evacuation response and reduce incident response times, resulting in improved aggregated patient outcomes 360. This approach allows evacuation assets to be treated as flexible dispatching nodes rather than static elements (e.g., a platoon or other grouping of crews or other resources). To avoid a combinatorial explosion, domain knowledge may be used to proactively prune search trees that fail to meet certain operational requirements.

MDPs, and/or various MDP subtypes such as partially observable MDPs (POMDPs), semi-MDPs (SMDPs), multi-agent MDPs (MMDPs), etc. capture the dynamics and uncertainties of complex systems and provide a principled manner of identifying and/or generating optimal or near-optimal policies. MDPs have an underlying structure that is appropriately informed by domain knowledge and can thus easily be amended for a particular application given the dynamic nature of the battlefield. Such an approach allows, for example, future integration of other vessel types (e.g., vertical lift aircraft) with different dynamics and constraints into the models and associated policies.

The three-level hierarchy of planners and associated MDPs has been developed specifically for military-type evacuation operations which have complex and intertwined command and control requirements, as well as aircrew and aircraft constraints that must be considered at scale. Unlike civilian ambulance EMS, which have one layer of dispatching nodes with typically homogeneous assets, military medical evacuation may include two layers of dispatching nodes-a theater medical planner or cell which allocates evacuation requests to evacuation platoons which then allocate those evacuation requests to certain aircraft and crews based on a set of myopic policies.

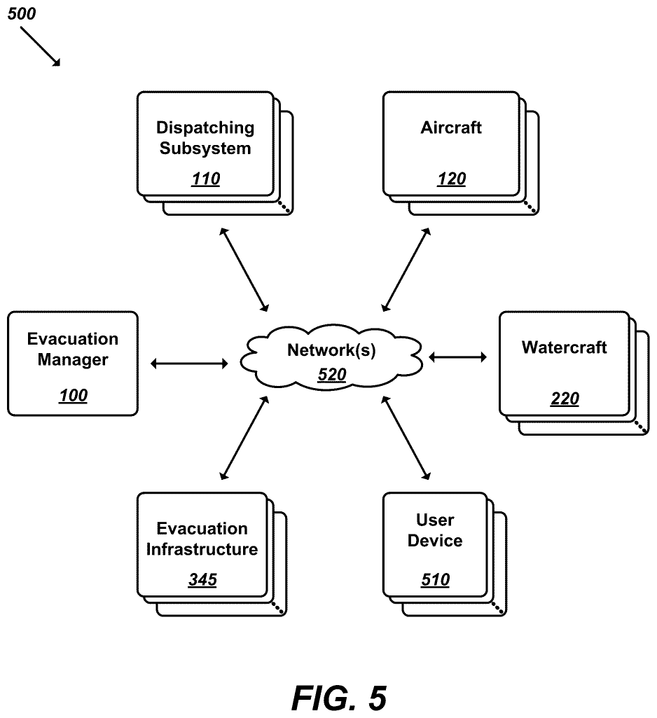

FIG. 5 illustrates a schematic block diagram of an environment 500 of one or more embodiments described herein. As shown, environment 500 may include evacuation manager 100, dispatching subsystems 110, aircraft 120, watercraft 220, evacuation infrastructure 345, user devices 510, network(s) 520, and/or other appropriate elements, components, devices, and/or systems.

Evacuation manager 100 may be, include, utilize, and/or otherwise be associated with a set of electronic components, devices, systems, and/or other appropriate elements. Evacuation manager 100 may include one or more processors that are able to execute instructions and/or otherwise manipulate data. Evacuation manager 100 may be able to communicate via networks 520. Evacuation manager 100 may be at least partially implemented using a device such as device 900 described below. Evacuation manager 100 may be able to direct, and/or otherwise utilize, components of other devices or systems. For instance, evacuation manager 100 may provide various GUIs via a display of user device 510. Evacuation manager 100 may implement a hierarchical framework, such as frameworks 300 or 400 Evacuation manager 100, and/or elements thereof, may be physically located with one or more dispatching subsystems 110, aircraft 120, watercraft 220, a remote ground station (not shown), and/or other appropriate location(s) where communication among elements of environment 500 is reliable and without detrimental latency.

Each dispatching subsystem 110 may be implemented using electronic devices, systems, and/or components that may be able to communicate across networks 520 and/or otherwise interact with the components of environment 500. A dispatching subsystem 110 may be associated with resources such as a platoon or other grouping of crews and evacuation assets.

Each aircraft 120, watercraft 220, ground-based vehicle, and/or other evacuation asset may be able to communicate via networks 520. Each aircraft 120, watercraft 220, ground-based vehicle, and/or other evacuation asset may be able to determine a current location, heading, status, capacity, and/or other relevant information, and may be able to communicate such information to other elements of environment 500.

Evacuation infrastructure 345 may include interfaces (e.g., an application programming interface (API)) and/or other communication pathways that may allow the elements of environment 500 to interact with the evacuation planners 350-355 and/or other evacuation infrastructure 345 resources.

Each user device 510 may be a device such as a smart phone, tablet, wearable device (e.g., a smart watch), and/or other type of device that allows user interaction with environment 500. User device 510 may be able to communicate via network 520. User device 510 may typically include various UI elements (e.g., a display, keypad, buttons, etc.) that may be used to provide information and/or instructions to a user and/or receive information and/or instructions from the user. In some cases, user devices 510 (and/or features described in association therewith) may be implemented via components of environment 500. For instance, a particular aircraft 120 may have various available UI element (e.g., displays, lights or other indicators, etc.) that may be utilized in a similar manner to a user device 510. User device 510 may be implemented using a device similar to device 900 described below.

Network(s) 520 may include various communication pathways, such as cellular networks, satellite-based systems, radio communication channels, optical communication channels, peer-to-peer communication channels, and/or any other available communication pathways. Information such as location information, evacuation requests, reallocation instructions, planning suggestions or recommendations, machine learning models, and/or other such information may be communicated across network(s) 520.

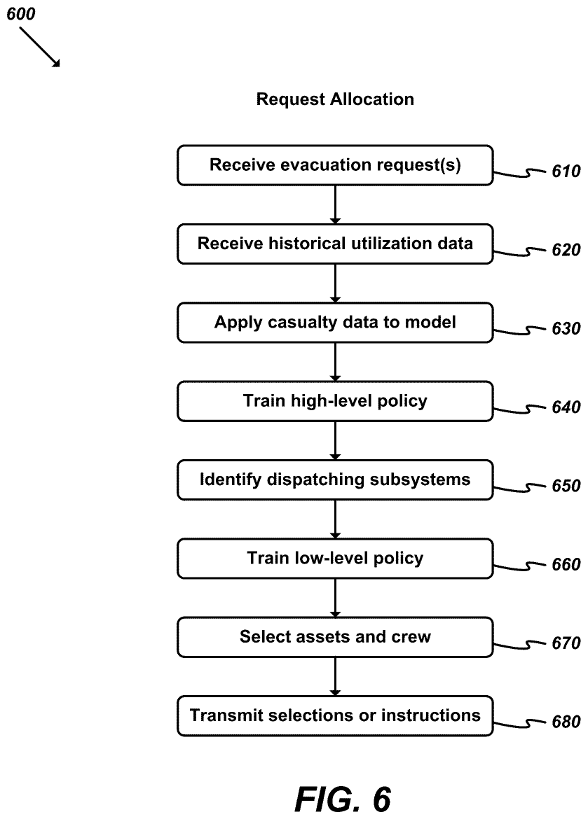

FIG. 6 illustrates an example process 600 for allocating evacuation requests. The process may be used to dynamically allocate and reallocate evacuation requests across available dispatching subsystems (e.g., dispatching subsystem 110). The process may be performed whenever evacuations operations are planned or implemented. In some embodiments, process 600 may be performed by evacuation manager 100.

As shown, process 600 may include receiving (at 610) an evacuation request, such as evacuation request 315. Evacuation requests 315 may be received at the evacuation manager 100 from various appropriate entities, such as dispatching subsystems 110, aircraft 120, watercraft 220, external or third-party entities, etc. Evacuation requests may be received via networks 520 and/or any other available channels of communication. Each evacuation request may include information such as location (and/or current speed, heading, etc.), type of request (e.g., indicating whether medical services are needed and/or which services are needed), identity of a requesting party, number of evacuees, and/or other relevant information. Received evacuation requests may be quantified in various appropriate ways. For example, in some embodiments an “evacuation demand” signal may be generated based on the received evacuation requests. Such a demand signal may be generated in various appropriate ways, such as by averaging evacuation request information over time. The demand signal may include, or be based on, request attributes such as location of requesting party, type of request, number of patients, patient status, etc.

Process 600 may include receiving (at 620) historical utilization data. Such utilization data may be received from each available dispatching subsystem 110 (as determined based on information such as location, status, type(s) and number(s) of evacuation assets, crew information, etc.). Utilization data may include data related to available evacuation assets (e.g., aircraft 120, watercraft 220, ground vehicles, etc.) such as type, number, status, etc. Utilization data may include data related to available crew such as type, number, status, etc.

The process may include applying (at 630) casualty data (e.g., observed casualty data, casualty estimates, etc.) to inform a casualty generation model, such as casualty generation model 325.

The process may include training (at 640) a high-level policy, such as high-level policy 335. The high-level policy 335 may be trained using casualty data generated using MDP models and simulations (e.g., as generated by casualty generation model 325). The high-level policy 335 may be trained with a goal related to aggregated patient outcome data, such as outcomes 360 (e.g., minimization of casualties, optimization of resource allocation, etc.).

Process 600 may include discretizing a list of feasible locations, based on an evacuation request (e.g., evacuation request 315) and evacuation asset attributes (e.g., evacuation asset attributes 305) that may serve as evacuation locations. Feasible locations may be identified in various appropriate ways based on various relevant attributes and/or parameters. For instance, feasible location regions, ranges, paths, and/or other types of areas may be identified based on evacuation asset capabilities and/or status (e.g., maximum or expected travel speed, current location, fuel, level, etc.). As another example, feasible location ranges may be identified based on environmental factors (e.g., weather, terrain, etc.). As still another example, feasible location ranges may be identified based on other attributes such as presence of adversarial entities, proximity to resources (e.g., refueling stations, medical facilities, etc.), and/or other relevant factors. The feasible location ranges may be converted to a listing of discrete locations, based on various relevant factors (e.g., navigation capability of transfer assets, available communication resources, etc.). For example, a region may be converted to a set of discrete points that are uniformly spaced about the region. The discretized list of feasible locations may be based at least partly on the transfer demand signal or similar information.

The process may include applying the discretized location list to the trained policy. Process 600 may include determining an optimal location, evacuation participants (e.g., dispatching subsystem(s) 110, participating aircraft 120 and/or watercraft 220, etc.), and/or other relevant evacuation attributes or parameters based on the application of the discretized location list to the trained policy. The optimal location may be determined in various appropriate ways. For instance, a score or metric may be generated for each location in the discretized location list and the locations may be sorted by score to determine the optimal location(s). Such a score or metric may be based on factors such as location of the dispatching subsystem 110, location, type, and/or status of associated evacuation assets, status of associated crew(s), the evacuation demand signal, and/or other relevant factors such as evacuation location attributes (e.g., geographic information indicating terrain, bodies of water, etc.), evacuation type, etc. A similar approach may be used to optimally select a set of evacuation assets to perform the evacuation(s).

As shown, process 600 may include identifying (at 650) dispatching subsystems, such as dispatching subsystem 110. Dispatching subsystems 110 may be identified in various appropriate ways. For instance, MDP models may be used to select from available dispatching subsystems 110. In some embodiments, a score or metric may be calculated for each available dispatching subsystem 110. Such a score or metric may be based on factors such as location of the dispatching subsystem 110, location, type, and/or status of associated evacuation assets, status of associated crew(s), and/or other relevant factors such as evacuation location attributes (e.g., geographic information indicating terrain, bodies of water, etc.), evacuation type, etc. The available dispatching subsystems 110 may be sorted based on the score or metric to identify the optimal dispatching subsystem(s) 110 to which to send an evacuation request.

Operations such as operations 610-660 may be performed by, via, and/or otherwise utilizing a resource such as high-level planner 420 and/or high-level planner 350 to allocate evacuation requests to dispatching subsystems 110.

The process may include training (at 660) a low-level policy, such as low-level policy 340. The low-level policy 340 may be trained using casualty data generated using MDP models and simulations (e.g., as generated by casualty generation model 325). The low-level policy 340 may be trained with a goal related to aggregated patient outcome data, such as outcomes 360 (e.g., minimization of casualties, optimization of resource allocation, etc.) and/or a goal related to local utilization of the dispatching subsystem 110 (and/or associated assets and/or crew).

As shown, process 600 may include selecting (at 670) assets and crew associated with one or more dispatching subsystems 110. Assets and crew may be selected in various appropriate ways. For instance, MDP models may be used to select from available assets and crew. In some embodiments, a score or metric may be calculated for each available asset and/or crew. Such a score or metric may be based on factors such as type and/or status of asset(s), status of associated crew(s), and/or other relevant factors such as evacuation location attributes (e.g., geographic information indicating terrain, bodies of water, etc.), evacuation type, etc. The available assets and/or crew may be sorted based on the score or metric to identify the optimal assets and/or crew to associate with each evacuation request.

Process 600 may include transmitting (at 680) selections and/or instructions. Such selections and/or instructions may be sent across channels such as network 520. Selections and/or instructions may include, for instance, identification of assets, identification of crews, instructions related to evacuation (e.g., navigation information), and/or other relevant information. In some embodiments, transmitting selections and/or instructions may include sending dispatch commands or instructions from the evacuation manager 100 to the evacuation assets via the dispatching subsystem 110 (or directly, and/or via other appropriate resources).

Operations such as operations 650-670 may be performed by, via, and/or otherwise utilizing a resource such as low-level planner 355 to determine optimal evacuation assets and/or crews for a given evacuation request.

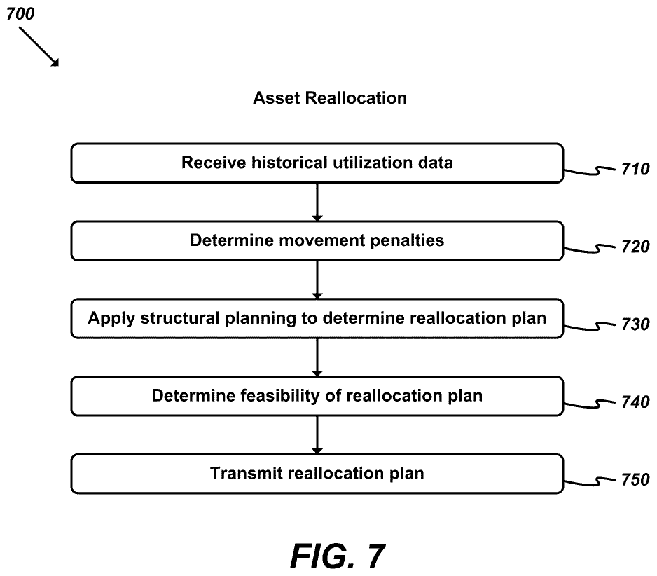

FIG. 7 illustrates an example process 700 for allocating evacuation assets. The process may be used to dynamically allocate and reallocate evacuation assets (e.g., aircraft 120, watercraft 220, ground vehicles, etc.) among available dispatching subsystems (e.g., dispatching subsystem 110). The process may be performed whenever evacuation operations are planned or implemented. In some embodiments, process 700 may be performed by evacuation manager 100.

As shown, process 700 may include receiving (at 710) utilization data. Such utilization data may be received from each available dispatching subsystem 110 (as determined based on information such as location, status, type(s) and number(s) of evacuation assets, crew information, etc.). Utilization data may include data related to available evacuation assets (e.g., aircraft 120, watercraft 220, ground vehicles, etc.) such as type, number, status, etc. Utilization data may include data related to available crew such as type, number, status, etc.

Process 700 may include determining (at 720) movement penalties for a selected set of reallocation procedures. The movement penalties may be determined using MDP models and simulations, based on, for example, data generated by casualty generation model 325.

The process may include applying (at 730) structural planning to determine a reallocation plan. Such structural planning may be implemented by, via, and/or otherwise utilizing a resource such as structural planner 410. Structural planning may include simulation of high-level planners 420 with penalties and generated casualty data (e.g., data generated by casualty generation model 325) to generate various sets of approximate outcomes 430.

As shown, process 700 may include determining (at 740) feasibility of the reallocation plan. The feasibility may be determined using MDP models and simulations, based on, for example, data generated by casualty generation model 325. In some embodiments, feasibility may be determined by determining a net benefit associated with reallocation (e.g., as indicated by a reduction in predicted casualties) and determining whether the net benefit exceeds a specified threshold. The threshold may be based on a system cost (e.g., time, utility, etc.) of the reallocation, where, for example, assets may not be performing evacuation-related operations as they are reallocated (e.g., as the assets move to a different location).

Process 700 may include transmitting (at 750) the reallocation plan including a set of reallocation procedures to the affected dispatching subsystems 110.

One of ordinary skill in the art will recognize that processes 600 and 700 may be implemented in various different ways without departing from the scope of the disclosure. For instance, the elements may be implemented in a different order than shown. As another example, some embodiments may include additional elements or omit various listed elements. Elements or sets of elements may be performed iteratively and/or based on satisfaction of some performance criteria. Non-dependent elements may be performed in parallel. Elements or sets of elements may be performed continuously and/or at regular intervals.

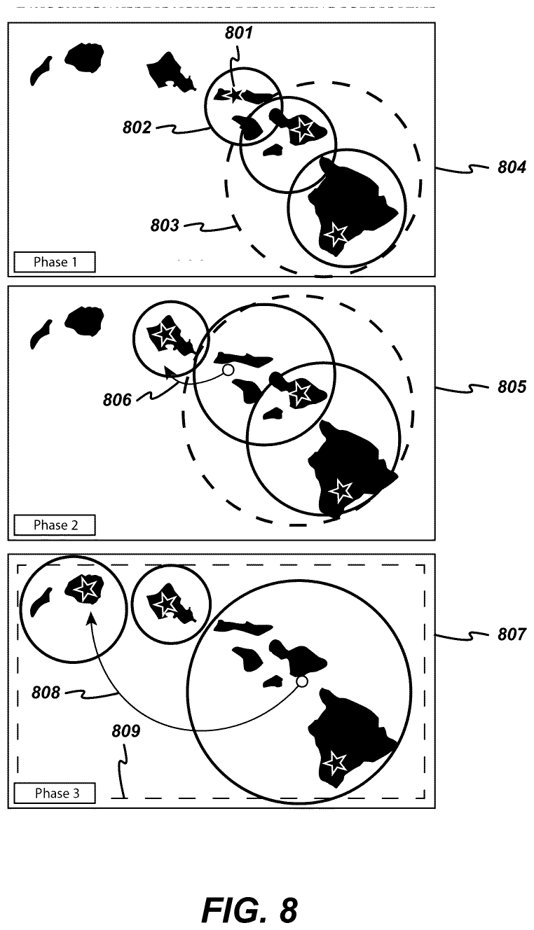

FIG. 8 illustrates another example overview of one or more embodiments described herein, in which exemplary evacuation operations are managed. In this example, a military campaign is being waged on the Hawaiian Islands across three subsequent operational phases. Three command nodes 801 are continuously jumped between islands, and two sets of range rings associated with different aircraft are adjusted in size, per guidance received from structural planner 410. Solid line range ring 802 represents the spatial area associated with a given command node for a short-range aircraft. Dashed line range ring 803 represents the spatial area associated with a given command node for a long-range aircraft. Each command node 801 may have a different composition of short-range and long-range aircraft that may evolve in time, again per guidance from structural planner 410. In Phase 1 804, all three command nodes 801 are situated on the eastern side of the Hawaiian Island chain. As the campaign progresses westward, casualties are generated on the westmost islands, and as seen in Phase 2 805, the range rings change in size as do the location of specific command nodes 801. Jump arrow 806 depicts the decision by structural planner 410 to jump a given command node 801 to the west to better respond to future casualties. Similarly, in Phase 3 807, jump arrow 808 depicts the decision of structural planner 410 to jump the intermediate command node to the westmost island, rather than the previously leftmost command node 801. This decision considers the increased penalty resulting from the longer jump distance against the benefit gained by the otherwise most optimal strategic location for each command node. Range box 809 demonstrates that the ranges associated with given evacuation assets may take any geometry appropriate to the geography considered.

The processes and modules described above may be at least partially implemented as software processes that may be specified as one or more sets of instructions recorded on a non-transitory storage medium. These instructions may be executed by one or more computational element(s) (e.g., microprocessors, microcontrollers, digital signal processors (DSPs), application-specific integrated circuits (ASICs), field programmable gate arrays (FPGAs), other processors, etc.) that may be included in various appropriate devices in order to perform actions specified by the instructions.

As used herein, the terms “computer-readable medium” and “non-transitory storage medium” are entirely restricted to tangible, physical objects that store information in a form that is readable by electronic devices.

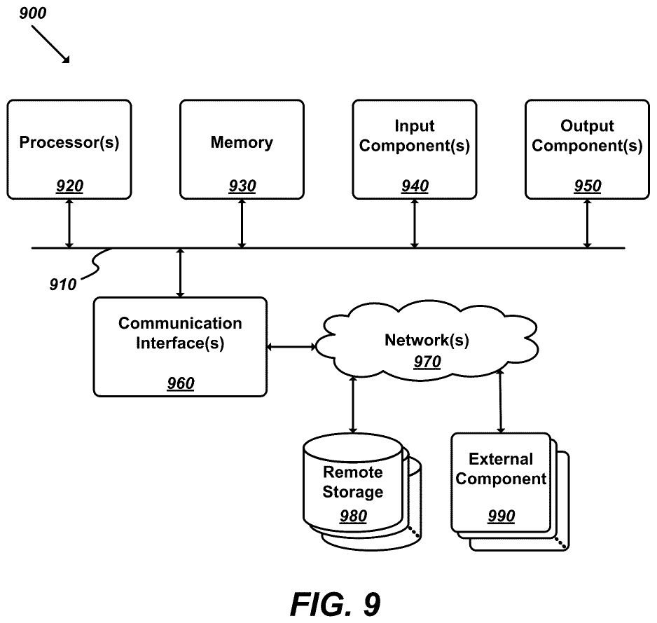

FIG. 9 illustrates a schematic block diagram of an exemplary device (or system or devices) 900 used to implement some embodiments. For example, the systems, devices, components, and/or operations described above in reference to FIG. 1, FIG. 2, FIG. 3, FIG. 4, and FIG. 5 may be at least partially implemented using device 900. As another example, the processes described in reference to FIG. 6 and FIG. 7 may be at least partially implemented using device 900.

Device 900 may be implemented using various appropriate elements and/or sub-devices. For instance, device 900 may be implemented using one or more personal computers (PCs), servers, mobile devices (e.g., smartphones), tablet devices, wearable devices, and/or any other appropriate devices. The various devices may work alone (e.g., device 900 may be implemented as a single smartphone) or in conjunction (e.g., some components of the device 900 may be provided by a mobile device while other components are provided by a server).

As shown, device 900 may include at least one communication bus 910, one or more processors 920, memory 930, input components 940, output components 950, and one or more communication interfaces 960.

Bus 910 may include various communication pathways that allow communication among the components of device 900. Processor 920 may include a processor, microprocessor, microcontroller, DSP, logic circuitry, and/or other appropriate processing components that may be able to interpret and execute instructions and/or otherwise manipulate data. Memory 930 may include dynamic and/or non-volatile memory structures and/or devices that may store data and/or instructions for use by other components of device 900. Such a memory device 930 may include space within a single physical memory device or spread across multiple physical memory devices.

Input components 940 may include elements that allow a user to communicate information to the computer system and/or manipulate various operations of the system. The input components may include keyboards, cursor control devices, audio input devices and/or video input devices, touchscreens, motion sensors, etc. Output components 950 may include displays, touchscreens, audio elements such as speakers, indicators such as light-emitting diodes (LEDs), printers, haptic or other sensory elements, etc. Some or all of the input and/or output components may be wirelessly or optically connected to the device 900.

Device 900 may include one or more communication interfaces 960 that are able to connect to one or more networks 970 or other communication pathways. For example, device 900 may be coupled to a web server on the Internet such that a web browser executing on device 900 may interact with the web server as a user interacts with an interface that operates in the web browser. Device 900 may be able to access one or more remote storages 980 and one or more external components 990 through the communication interface 960 and network 970. The communication interface(s) 960 may include one or more application programming interfaces (APIs) that may allow the device 900 to access remote systems and/or storages and also may allow remote systems and/or storages to access device 900 (or elements thereof).

It should be recognized by one of ordinary skill in the art that any or all of the components of computer system 900 may be used in conjunction with some embodiments.

Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

Device 900 may perform various operations in response to processor 920 executing software instructions stored in a computer-readable medium, such as memory 930. Such operations may include manipulations of the output components 950 (e.g., display of information, haptic feedback, audio outputs, etc.), communication interface 960 (e.g., establishing a communication channel with another device or component, sending and/or receiving sets of messages, etc.), and/or other components of device 900.

The software instructions may be read into memory 930 from another computer-readable medium or from another device. The software instructions stored in memory 930 may cause processor 920 to perform processes described herein. Alternatively, hardwired circuitry and/or dedicated components (e.g., logic circuitry, ASICs, FPGAs, etc.) may be used in place of or in combination with software instructions to implement processes described herein. Thus, implementations described herein are not limited to any specific combination of hardware circuitry and software.

The actual software code or specialized control hardware used to implement an embodiment is not limiting of the embodiment. Thus, the operation and behavior of the embodiment has been described without reference to the specific software code, it being understood that software and control hardware may be implemented based on the description herein.

While certain connections or devices are shown, in practice additional, fewer, or different connections or devices may be used. Furthermore, while various devices and networks are shown separately, in practice the functionality of multiple devices may be provided by a single device or the functionality of one device may be provided by multiple devices. In addition, multiple instantiations of the illustrated networks may be included in a single network, or a particular network may include multiple networks. While some devices are shown as communicating with a network, some such devices may be incorporated, in whole or in part, as a part of the network.

Some implementations are described herein in conjunction with thresholds. To the extent that the term “greater than” (or similar terms) is used herein to describe a relationship of a value to a threshold, it is to be understood that the term “greater than or equal to” (or similar terms) could be similarly contemplated, even if not explicitly stated. Similarly, to the extent that the term “less than” (or similar terms) is used herein to describe a relationship of a value to a threshold, it is to be understood that the term “less than or equal to” (or similar terms) could be similarly contemplated, even if not explicitly stated. Further, the term “satisfying,” when used in relation to a threshold, may refer to “being greater than a threshold,” “being greater than or equal to a threshold,” “being less than a threshold,” “being less than or equal to a threshold,” or other similar terms, depending on the appropriate context.

No element, act, or instruction used in the present application should be construed as critical or essential unless explicitly described as such. An instance of the use of the term “and,” as used herein, does not necessarily preclude the interpretation that the phrase “and/or” was intended in that instance. Similarly, an instance of the use of the term “or,” as used herein, does not necessarily preclude the interpretation that the phrase “and/or” was intended in that instance. Also, as used herein, the article “a” is intended to include one or more items and may be used interchangeably with the phrase “one or more.” Where only one item is intended, the terms “one,” “single,” “only,” or similar language is used. Further, the phrase “based on” is intended to mean “based, at least in part, on” unless explicitly stated otherwise.

The foregoing relates to illustrative details of exemplary embodiments and modifications may be made without departing from the scope of the disclosure. Even though particular combinations of features are recited in the claims and/or disclosed in the specification, these combinations are not intended to limit the possible implementations of the disclosure. In fact, many of these features may be combined in ways not specifically recited in the claims and/or disclosed in the specification. For instance, although each dependent claim listed below may directly depend on only one other claim, the disclosure of the possible implementations includes each dependent claim in combination with every other claim in the claim set.

1. A device, comprising:
one or more processors configured to:
receive a set of evacuation requests;
identify dispatching subsystems associated with the set of evacuation requests, each dispatching subsystem associated with a set of evacuation assets;
apply structural planning to generate a reallocation plan based on the set of evacuation requests, the associated dispatching subsystems, and the sets of evacuation assets;
calculate, based on the structural planning, a net benefit of reallocation;
determine that the net benefit of reallocation exceeds a specified threshold; and
reallocate one or more evacuation assets from the sets of evacuation assets among the associated dispatching subsystems.

2. The device of claim 1, wherein reallocating one or more evacuation assets from the sets of evacuation assets among the associated dispatching subsystems comprises providing reallocation instructions to a particular dispatching subsystem from the associated dispatching subsystems.

3. The device of claim 2, wherein the evacuation assets comprise a set of evacuation sub-assets and the particular dispatching subsystem provides evacuation instructions to the set of evacuation sub-assets.

4. The device of claim 3, wherein the evacuation assets comprise airborne, waterborne, or land-based assets.

5. The device of claim 1, wherein calculating the net benefit of reallocation comprises training an asset reallocation policy using reinforcement learning techniques based on generated casualty data applied via simulations and evacuation asset attributes including location, speed, and patient carrying capacity.

6. The device of claim 5, wherein the asset reallocation policy is trained using historical utilization data of evacuation assets, a casualty generation model, and the set of evacuation asset attributes.

7. The device of claim 6, wherein training the asset reallocation policy, training a request allocation high-level policy, and training an asset dispatch low-level policy comprises utilizing Markov decision process (MDP) models and simulated casualty data.

8. A non-transitory computer-readable medium, storing a plurality of processor-executable instructions to:
receive a set of evacuation requests;
identify dispatching subsystems associated with the set of evacuation requests, each dispatching subsystem associated with a set of evacuation assets;
apply structural planning to generate a reallocation plan based on the set of evacuation requests, the associated dispatching subsystems, and the sets of evacuation assets;
calculate, based on the structural planning, a net benefit of reallocation;
determine that the net benefit of reallocation exceeds a specified threshold; and
reallocate one or more evacuation assets from the sets of evacuation assets among the associated dispatching subsystems.

9. The non-transitory computer-readable medium of claim 8, wherein reallocating one or more evacuation assets from the sets of evacuation assets among the associated dispatching subsystems comprises providing reallocation instructions to a particular dispatching subsystem from the associated dispatching subsystems.

10. The non-transitory computer-readable medium of claim 9, wherein the evacuation assets comprise a set of evacuation sub-assets and the particular dispatching subsystem provides evacuation instructions to the set of evacuation sub-assets.

11. The non-transitory computer-readable medium of claim 10, wherein the evacuation assets comprise airborne, waterborne, or land-based assets.

12. The non-transitory computer-readable medium of claim 8, wherein calculating the net benefit of reallocation comprises training an asset reallocation policy using reinforcement learning techniques based on generated casualty data applied via simulations and evacuation asset attributes including location, speed, and patient carrying capacity.

13. The non-transitory computer-readable medium of claim 12, wherein the asset reallocation policy is trained using historical utilization data of evacuation assets, a casualty generation model, and the set of evacuation asset attributes.

14. The non-transitory computer-readable medium of claim 13, wherein training the asset reallocation policy, training a request allocation high-level policy, and training an asset dispatch low-level policy comprises utilizing Markov decision process (MDP) models and simulated casualty data.

15. A method comprising:
receiving a set of evacuation requests;
identifying dispatching subsystems associated with the set of evacuation requests, each dispatching subsystem associated with a set of evacuation assets;
applying structural planning to generate a reallocation plan based on the set of evacuation requests, the associated dispatching subsystems, and the sets of evacuation assets;
calculating, based on the structural planning, a net benefit of reallocation;
determining that the net benefit of reallocation exceeds a specified threshold; and
reallocating one or more evacuation assets from the sets of evacuation assets among the associated dispatching subsystems.

16. The method of claim 15, wherein reallocating one or more evacuation assets from the sets of evacuation assets among the associated dispatching subsystems comprises providing reallocation instructions to a particular dispatching subsystem from the associated dispatching subsystems.

17. The method of claim 16, wherein the evacuation assets comprise a set of evacuation sub-assets and the particular dispatching subsystem provides evacuation instructions to the set of evacuation sub-assets.

18. The method of claim 17, wherein the evacuation assets comprise airborne, waterborne, or land-based assets.

19. The method of claim 15, wherein calculating the net benefit of reallocation comprises training an asset reallocation policy using reinforcement learning techniques based on generated casualty data applied via simulations and evacuation asset attributes including location, speed, and patient carrying capacity.

20. The method of claim 19, wherein the asset reallocation policy is trained using historical utilization data of evacuation assets, a casualty generation model, and the set of evacuation asset attributes, and wherein training the asset reallocation policy, training a request allocation high-level policy, and training an asset dispatch low-level policy comprises utilizing Markov decision process (MDP) models and simulated casualty data.

21. A device comprising a communication interface and one or more processors configured to:
receive from each of a plurality of dispatching subsystems utilization data indicating locations and statuses of the dispatching subsystems and types, numbers, and statuses of evacuation assets and crews associated with the respective dispatching subsystems;
determine movement penalties for a set of reallocation procedures using one or more Markov decision process models and simulations based on casualty data generated by a casualty-generation model;
apply a structural planner to generate a reallocation plan by simulating a plurality of high-level planners using the movement penalties and the generated casualty data to generate respective approximate outcomes;
calculate a net benefit of the reallocation plan, the net benefit being indicated by a reduction in predicted casualties;
determine that the net benefit exceeds a threshold based on a time or utility cost incurred while an evacuation asset is unavailable during reallocation;
reallocate at least one evacuation asset among the plurality of dispatching subsystems; and
transmit the reallocation plan, including at least one of the reallocation procedures, to affect dispatching subsystems.

22. The device of claim 21 wherein a high-level planner allocates an evacuation request to one of the dispatching subsystems using a request-allocation high-level policy, and a low-level planner associated with the allocated dispatching subsystem selects an evacuation asset and crew using an asset-dispatch low-level policy.
